(19) United States
(12) Patent Application Publication
(10) Pub. No.: US 2014/0188203 A1
(43) Pub. Date: Jul. 3, 2014

(54) HANDLE ASSEMBLY FOR MEDICAL DEVICE DEPLOYMENT SYSTEM

(71) Applicants: Brent Mayle, Spencer, IN (US); James Merk, Terre Haute, IN (US); Jeffry S. Melsheimer, Springville, IN (US)

(72) Inventors: Brent Mayle, Spencer, IN (US); James Merk, Terre Haute, IN (US); Jeffry S. Melsheimer, Springville, IN (US)

(21) Appl. No.: 13/729,471

(22) Filed: Dec. 28, 2012

(51) Int. Cl. A61F 2/95

(52) U.S. Cl. CPC A61F 2/95
USPC 623/1.11

(57) ABSTRACT

A medical device deployment system includes an inner shaft telescopically received within a retractable sheath and a medical device positioned over the inner shaft. A handle assembly includes a proximal handle portion having a handle body having an elongate slot therethrough. A distal end of the handle body is attached to an open proximal end of the retractable sheath. The handle assembly also includes a distal handle portion attached to a proximal end of the inner shaft through a connection portion. In a pre-deployment configuration, the connection portion is positioned through a proximal end of the elongate slot and the retractable sheath restricts radial expansion of the medical device. In a post-deployment configuration, the connection portion is positioned through a distal end of the elongate slot and the retractable sheath is retracted such that the medical device is not restricted from radial expansion by the retractable sheath.

### TECHNICAL FIELD

The present disclosure relates generally to a handle assembly for a medical device deployment system, and more particularly to a handle assembly including a proximal handle portion and a distal handle portion axially movable relative to one another to deploy a medical device.

### BACKGROUND

Various medical devices, including stents, stent grafts, and venous filters, are deployed within the vasculature of a patient using deployment devices. Some of the medical devices are self-expanding, in a radial direction, and require restriction from radial expansion prior to deployment. According to some deployment systems, an outer sheath maintains a restricted position of the self-expanding medical device during advancement of the medical device to a deployment site. Once the medical device is positioned at or near the deployment site, the sheath is removed, or retracted, to permit radial expansion of the self-expanding medical device. The retraction of the sheath is typically facilitated through manipulation of a handle positioned at a proximal end of the deployment system.

Although a variety of different deployment system handles exist, ranging from relatively simple to relatively complex devices, a conventional pull-type handle includes a proximal handle portion and a distal handle portion. The proximal handle portion is configured to maintain a relatively stationary position of a pusher catheter, which supports the self-expanding medical device, while the distal handle portion is configured to retract a sheath positioned over the self-expanding medical device. Prior to deployment, the proximal and distal handle portions are positioned away from one another, resulting in a relatively long pre-deployment length of the deployment system. Deployment of the medical device is initiated by proximally retracting the distal handle portion, which is connected to the sheath, toward the proximal handle portion, which is connected to the pusher catheter. In addition to having a relatively long pre-deployment length, the conventional pull-type handle requires non-intuitive manipulation of the handle portions, which may be difficult for less experienced clinicians. Further, the expanded pre-deployment configuration may be relatively unstable, particularly for the deployment of relatively large medical devices.

U.S. Patent Application Publication No. 2007/0219617 to Saint discloses a handle for a long self expanding stent. In particular, the handle includes a housing and a spool. A pushrod has a proximal end connected to the housing of the handle. A retraction wire is connected to a proximal end of a sheath and to the spool. Retraction of the sheath is accomplished by winding the refraction wire around the spool. Accordingly, the handle may be shorter than the stent. Although the handle of the Saint disclosure may be useful for some applications, it should be appreciated that there is a continuing need for efficient and effective handles for medical device deployment systems.

The present disclosure is directed toward one or more of the problems or issues set forth above.

### SUMMARY OF THE DISCLOSURE

In one aspect, a medical device deployment system includes an inner shaft having an elongate body, a proximal end, a distal end, and a medical device support region at the distal end of the elongate body. The inner shaft is telescopically received within a retractable sheath having an elongate tubular body defining a lumen extending from an open proximal end to an open distal end. A medical device is positioned over the inner shaft at the medical device support region. A handle assembly includes a proximal handle portion having a handle body having an elongate slot therethrough. A distal end of the handle body is attached to the open proximal end of the retractable sheath. The handle assembly also includes a distal handle portion attached to the proximal end of the inner shaft through a connection portion, which is positioned through and movable within the elongate slot. The medical device deployment system has a pre-deployment configuration in which the connection portion is positioned through a proximal end of the elongate slot and the retractable sheath restricts radial expansion of the medical device. The medical device deployment system also has a post-deployment configuration in which the connection portion is positioned through a distal end of the elongate slot and the retractable sheath is retracted such that the medical device is not restricted from radial expansion by the retractable sheath.

In another aspect, a method of deploying a medical device within a body lumen using the medical device deployment system described above is also provided. The method includes a step of advancing a distal portion of the medical device deployment system toward a deployment site within the body lumen with the medical device deployment system in the pre-deployment configuration, in which the connection portion is positioned through a proximal end of the elongate slot and the retractable sheath restricts radial expansion of the medical device. The method also includes proximally retracting the retractable sheath by proximally retracting the proximal handle portion while maintaining a stationary position of the distal handle portion relative to the proximal handle portion. The medical device is then radially expanded responsive to the proximally retracting step.

### BRIEF DESCRIPTION OF THE DRAWINGS

FIG. 1 is a perspective view of a medical device deployment system, according to one embodiment of the present disclosure;

FIG. 2 is a partially sectioned side diagrammatic view of the medical device deployment system of FIG. 1, shown in a pre-deployment configuration;

FIG. 3 is a partially sectioned side diagrammatic view of the medical device deployment system of the previous Figs., shown in a post-deployment configuration;

FIG. 4 is a side diagrammatic view of a vascular structure of a patient at one stage of a medical device deployment procedure using the medical device deployment system of the previous Figs.;

FIG. 5 is a side diagrammatic view of the vascular structure at another stage of the medical device deployment procedure; and

FIG. 6 is a side diagrammatic view of the vascular structure at another stage of the medical device deployment procedure.

### DETAILED DESCRIPTION

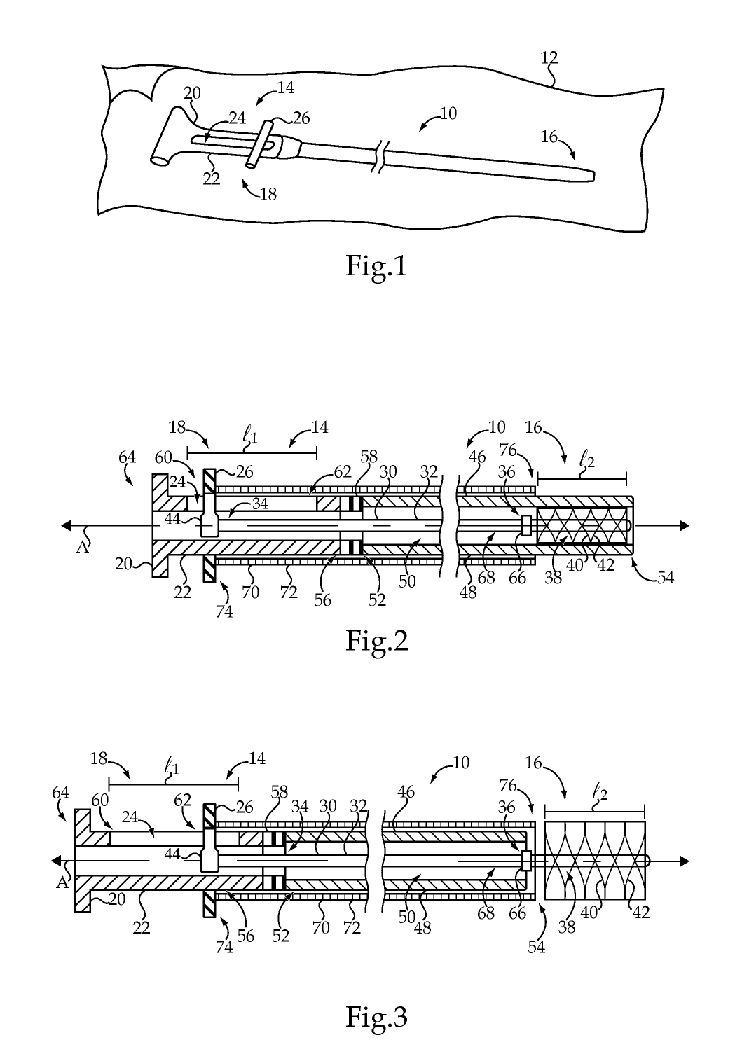

Referring to FIG. 1, there is shown a medical device deployment system 10 according to one embodiment of the present disclosure. The medical device deployment system 10 may include a number of components, which may be provided within a sterile, tear open package 12, as is known in the art. In performing a medical device deployment procedure on a patient, some or all of the components of the medical device deployment system 10 may be used, depending upon the specifics of the procedure to be performed. As should be appreciated, however, components of the medical device deployment system 10 might be separately packaged and/or the medical device deployment system 10 might also include components in addition to those shown, including components routinely used in percutaneous vascular procedures.

In general, the medical device deployment system 10 has a proximal end 14 and a distal end 16. As shown, a handle assembly 18, which may include relatively rigid components made from medical grade materials, is disposed at the proximal end 14. The handle assembly 18 generally includes a proximal handle portion 20 having a handle body 22 with an elongate slot 24 therethrough and a distal handle portion 26. As will be described in greater detail below, the proximal handle portion 20 and the distal handle portion 26 are movable relative to one another during a medical device deployment procedure. In the present disclosure, “proximal” will be used to refer to the end of a component or feature that is closest to a clinician, while “distal” is used to refer to a component or feature that is farthest away from the clinician. Such meanings are consistent with conventional use of the terms and, as such, should be understood by those skilled in the art.

Turning now to FIGS. 2 and 3, the medical device deployment system 10, according to an exemplary embodiment, will be discussed in greater detail. The medical device deployment system 10 includes an inner shaft 30 having an elongate body 32, a proximal end 34, a distal end 36, and a medical device support region 38 at the distal end 36 of the elongate body 32. According to some embodiments, the inner shaft 30, which may include a hollow tubular body, may range in length from several inches to several feet long, and may have a catheter wall diameter that is orders of magnitude smaller than its length. The elongate body 32 may be made from any common medical tube material, such as, for example, a plastic, rubber, silicone, or Teflon material, and may exhibit both firmness and flexibility.

A medical device 40 may be positioned over the inner shaft 30 at the medical device support region 38. According to the exemplary embodiment, the medical device 40 may include a radially expanding stent 42 for providing tubular support within a blood vessel, canal, duct, or other bodily passageway. Radially expandable stents 42 are known and may be expanded using a balloon, or other known device, positioned at a distal portion of a delivery catheter. Alternatively, and according to the exemplary embodiment, the radially expanding stent 42 may be made from a resilient or shape memory material, such as, for example, nitinol, that is capable of self-expanding from a compressed state to an expanded state without the application of a radial force on the stent 42. Such a stent 42 may be referred to as a “self-expanding” stent 42. Although a self-expanding stent 42 will be discussed herein, those skilled in the art should appreciate that the medical device 40 may include alternative radially expandable prosthetic implants. For example, the medical device 40 may include a self-expanding, or otherwise expandable, stent graft or venous filter.

The proximal end 34 of the inner shaft 30 may be attached to the distal handle portion 26 through a connection portion 44. The connection portion 44, which may be attached to or integral with one or both of the inner shaft 30 and the distal handle portion 26, is positioned through and movable within the elongate slot 24. The connection portion 44 may be solid or hollow, as necessary, depending on the requirements for the medical device deployment procedure. It should be appreciated that connections or attachments between components of the catheter assembly 10 may be made using attachment means known to those skilled in the art.

A retractable sheath 46 has an elongate tubular body 48 defining a lumen 50 extending from an open proximal end 52 to an open distal end 54. As shown, the inner shaft 30 is telescopically received within the retractable sheath 46. When the self-expanding stent 42, or other medical device, is loaded onto the inner shaft 30, the self-expanding stent 42 may be restricted from self-expansion using the elongate tubular sheath 46, which is slidably received over the elongate tubular body 32 of the inner shaft 30. According to this configuration, the retractable sheath 46 restricts radial expansion of the self-expanding stent 42 by contacting the stent 42 with an inner wall surface defining the lumen 50 of the retractable sheath 46. The open proximal end 52 of the retractable sheath 46 is attached to a distal end 56 of the handle body 22, such as through an adaptor 58, or other similar device or connector.

The medical device deployment system 10 has a pre-deployment configuration, as shown in FIG. 2, in which the connection portion 44 is positioned through a proximal end 60 of the elongate slot 24 and the retractable sheath 46 restricts radial expansion of the medical device 40. The medical device deployment system 10 also has a post-deployment configuration, as shown in FIG. 3, in which the connection portion 44 is positioned through a distal end 62 of the elongate slot 24 and the retractable sheath 46 is retracted such that the medical device 40 is not restricted from radial expansion by the retractable sheath 46. To facilitate movement of the medical device deployment system 10 between the pre-deployment configuration of FIG. 2 and the post-deployment configuration of FIG. 3, a clinician may maintain a stationary position of the distal handle portion 26 while axially moving, relative to axis A, a proximal end 64 of the proximal handle portion 20.

An axial length L₁ of the elongate slot 24 may correspond to, or be at least as long as, an axial length of L₂ of the medical device support region 38. According to some embodiments, an axial length of the self-expanding stent 42, which may correspond to axial length L₂, may be greater than about 100 mm. According to some embodiments, the medical device 40 may have an axial length of between about 10 mm and 300 mm. More particularly, the medical device 40 may range from 20 mm in length to 200 mm in length. It should be appreciated that such dimensions are provided for exemplary purposes only, and a variety of medical devices, having various sizes and configurations, may be deployed using the medical device deployment system 10 described herein.

According to some embodiments, a pusher band 66 may be disposed on an exterior 68 of the inner shaft 30 proximal to the medical device 40 and may be configured to restrict proximal movement of the medical device 40 during relative movement of the inner shaft 30 and the retractable sheath 46. According to some embodiments, and as shown in FIGS. 2 and 3, the medical device deployment system 10 may also include a stability sheath 70 having an elongate tubular body 72 having an open proximal end 74 and an open distal end 76. The retractable sheath 46 may be telescopically received within the stability sheath 70 and the distal handle portion 26 may be attached to the open proximal end 74 of the stability sheath 70.

### INDUSTRIAL APPLICABILITY

The present disclosure is generally applicable to medical device deployment systems. More specifically, the present disclosure is applicable to deployment systems for deploying self-expanding medical devices, such as stents, grafts, filters, and the like. Further, the present disclosure is applicable to handle assemblies for use with such medical device deployment devices.

Referring generally to FIGS. 1-6, a medical device deployment system 10 includes an inner shaft 30 telescopically received within a retractable sheath 46 and a medical device 40 positioned over the inner shaft 30. A handle assembly 18 includes a proximal handle portion 20 having a handle body 22 with an elongate slot 24 therethrough. A distal end 56 of the handle body 22 is attached to an open proximal end 52 of the retractable sheath 46. The handle assembly 18 also includes a distal handle portion 26 attached to a proximal end 34 of the inner shaft 30 through a connection portion 44.

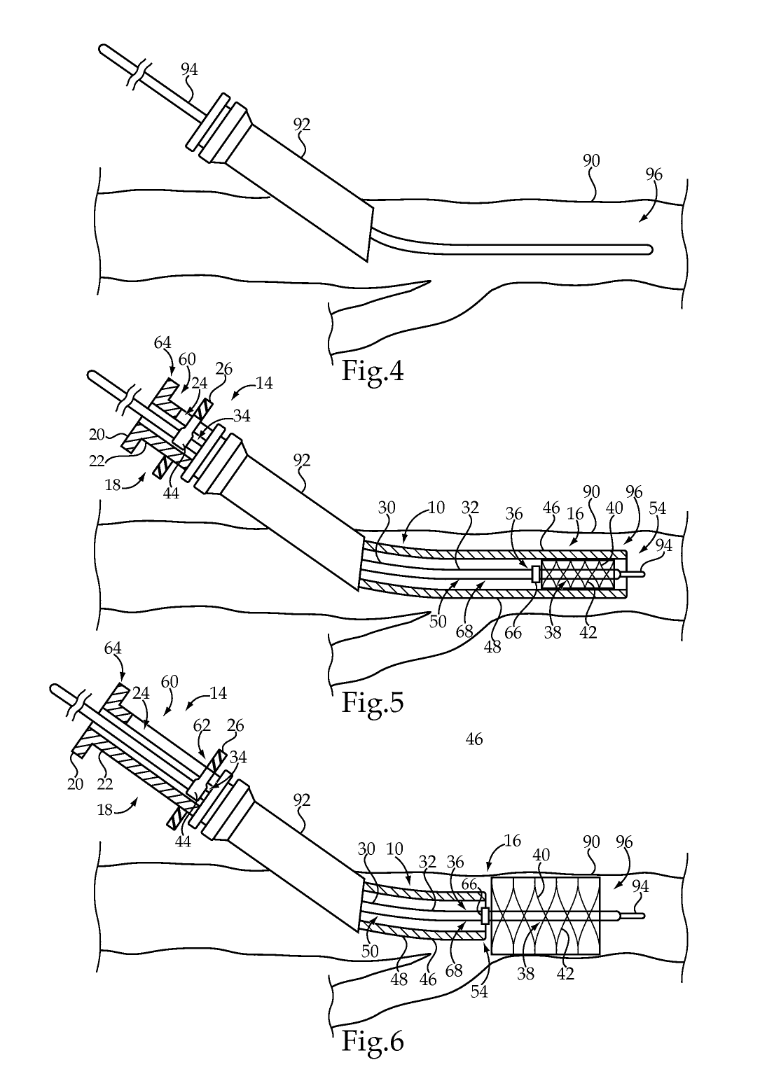

Referring specifically to FIGS. 4 through 6, a percutaneous vascular procedure using the medical device deployment system 10 of the previous Figs. will be discussed with reference to a vascular structure 90 of a patient. The vascular structure 90, as should be appreciated, may include a vessel wall defining a lumen. A clinician may first position a needle, or introducer 92, through the skin of a patient to gain access to the vascular structure 90. At a next stage of the procedure, a clinician may insert a conventional wire guide 94 through a tube of the introducer and into the vascular structure 90, as shown in FIG. 4.

Turning now to FIG. 5, a distal portion 16 of the medical device deployment system 10 may be inserted through the introducer 92 and over the wire guide 94. In particular a hollow elongate body 32 of the inner shaft 30 may be advanced over the wire guide 94. A distal portion 16 of the medical device deployment system 10 may be advanced toward a deployment site 96 within the body lumen 90 with the medical device deployment system 10 in a pre-deployment configuration, as described with respect to FIG. 2, in which the connection portion 44 is positioned through a proximal end 60 of the elongate slot 24 in the retractable sheath 46 restricts radial expansion of the medical device 40. With the medical device deployment system 10 properly positioned relative to the deployment site 96, the retractable sheath 46 may be proximally refracted by proximally moving, or retracting, the proximal handle portion 20 while maintaining a stationary position of the distal handle portion 26 relative to the proximal handle portion 20. The medical device 40, which may include a self-expanding stent 42, may be radially expanded responsive to the retraction of the retractable sheath 46, as shown in FIG. 6.

As described above, retraction of the retractable sheath 46 and, thus, deployment of the medical device 40 includes moving the proximal handle portion 20 relative to the distal handle portion 26 such that the connection portion 44 is positioned through the distal end 62 of the elongate slot 24. As should be appreciated, this may include proximally refracting the proximal handle portion 20 an axial distance corresponding to an axial length L₂ of the medical device 40 and/or medical device support region 38. A pusher band 66 may be disposed on an exterior 68 of the inner shaft 30 proximal to the medical device 40 for restricting proximal movement of the medical device 40 during the proximally retracting step. According to some embodiments, such as those shown in FIGS. 2 and 3, the proximally retracting step may include telescopically moving the retractable sheath 46 within a stability sheath 70 having an open proximal end 74 attached to the distal handle portion 26. After deployment of the medical device 40, the medical device deployment system 10 may be withdrawn from the body lumen 90 with the medical device deployment system 10 in a post-deployment configuration, as described with reference to FIG. 3, in which the connection portion 44 is positioned through a distal end 62 of the elongate slot 24.

The medical device deployment system 10 described herein provides an effective means for deploying medical devices 40 of various sizes and configurations. The handle assembly 18 of the medical device deployment system has a relatively short pre-deployment length, particularly when compared to conventional pull-type handles, and includes an intuitive manipulation of the handle portions 20 and 26 (i.e., proximally retracting the proximal handle portion 20 results in proximal retraction of the retractable sheath 46). Further, the shorter pre-deployment configuration may provide more stability for the deployment of medical devices 40, including relatively large medical devices.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A medical device deployment system, comprising:
an inner shaft having an elongate body, a proximal end, a distal end, and a medical device support region at the distal end of the elongate body;
a retractable sheath having an elongate tubular body defining a lumen extending from an open proximal end to an open distal end, wherein the inner shaft is telescopically received within the retractable sheath;
a medical device positioned over the inner shaft at the medical device support region; and
a handle assembly including:
a proximal handle portion having a handle body having an elongate slot therethrough, wherein a distal end of the handle body is attached to the open proximal end of the retractable sheath; and
a distal handle portion attached to the proximal end of the inner shaft through a connection portion, wherein the connection portion is positioned through and movable within the elongate slot;
wherein the medical device deployment system has a pre-deployment configuration in which the connection portion is positioned through a proximal end of the elongate slot and the retractable sheath restricts radial expansion of the medical device, and a post-deployment configuration in which the connection portion is positioned through a distal end of the elongate slot and the retractable sheath is retracted such that the medical device is not restricted from radial expansion by the retractable sheath.

2. The medical device deployment system of claim 1, wherein an axial length of the elongate slot is at least as long as an axial length of the medical device support region.

3. The medical device deployment system of claim 1, wherein the elongate body of the inner shaft is hollow.

4. The medical device deployment system of claim 3, further including a pusher band disposed on an exterior of the inner shaft proximal to the medical device and configured to restrict proximal movement of the medical device during relative movement of the inner shaft and the retractable sheath.

5. The medical device deployment system of claim 4, wherein the medical device is a self-expanding stent.

6. The medical device deployment system of claim 5, wherein an axial length of the self-expanding stent is greater than about 100 millimeters.

7. The medical device deployment system of claim 3, further including a wire guide positioned through at least a portion of the elongate body of the inner shaft.

8. The medical device deployment system of claim 1, further including a stability sheath having an elongate tubular body having an open proximal end and an open distal end, wherein the retractable sheath is telescopically received within the stability sheath and the distal handle portion is attached to the open proximal end of the stability sheath.

9. A method of deploying a medical device within a body lumen using a medical device deployment system, wherein the medical device deployment system includes: an inner shaft having an elongate body, a proximal end, a distal end, and a medical device support region at the distal end of the elongate body; a retractable sheath having an elongate tubular body defining a lumen extending from an open proximal end to an open distal end, wherein the inner shaft is telescopically received within the retractable sheath; the medical device being positioned over the inner shaft at the medical device support region; and a handle assembly including a proximal handle portion having a handle body having an elongate slot therethrough, wherein a distal end of the handle body is attached to the open proximal end of the retractable sheath, and a distal handle portion attached to the proximal end of the inner shaft through a connection portion, wherein the connection portion is positioned through and movable within the elongate slot, the method comprising steps of:
advancing a distal portion of the medical device deployment system toward a deployment site within the body lumen with the medical device deployment system in a pre-deployment configuration in which the connection portion is positioned through a proximal end of the elongate slot and the retractable sheath restricts radial expansion of the medical device;
proximally retracting the retractable sheath by proximally retracting the proximal handle portion while maintaining a stationary position of the distal handle portion relative to the proximal handle portion; and
radially expanding the medical device responsive to the proximally retracting step.

10. The method of claim 9, further including withdrawing the medical device deployment system from the body lumen with the medical device deployment system in a post-deployment configuration in which the connection portion is positioned through a distal end of the elongate slot.

11. The method of claim 9, wherein the proximally retracting step includes moving the proximal handle portion relative to the distal handle portion such that the connection portion is positioned through a distal end of the elongate slot.

12. The method of claim 11, wherein the proximally retracting step includes proximally retracting the proximal handle portion an axial distance at least as long as an axial length of the medical device support region.

13. The method of claim 12, further including restricting proximal movement of the medical device during the proximally retracting step using a pusher band disposed on an exterior of the inner shaft proximal to the medical device.

14. The method of claim 12, wherein the radially expanding step includes radially expanding a self-expanding stent.

15. The method of claim 14, wherein the radially expanding step includes radially expanding a self-expanding stent having an axial length greater than about 100 millimeters.

16. The method of claim 11, wherein the advancing step includes advancing at least a portion of a hollow elongate body of the inner shaft over a wire guide.

17. The method of claim 11, further including inserting the distal portion of the medical device deployment system through an introducer prior to the advancing step.

18. The method of claim 17, wherein the proximally retracting step includes telescopically moving the retractable sheath within a stability sheath having an open proximal end attached to the distal handle portion.
